(19) United States
(12) Patent Application Publication
(10) Pub. No.: US 2005/0113856 A1
(43) Pub. Date: May 26, 2005

(54) Method of joining materials

(76) Inventors: Adam S. Epstein, Wellesley, MA (US); Stephen J. Wiater, Southwick, MA (US); Brian Creston, Milford, CT (US)

Correspondence Address: Mark Farber, Esq.;United States Surgical, A Division of Tyco Healthcare Group, LP, 150 Glover Avenue, Norwalk, CT, 06856, US

(21) Appl. No.: 10/966,419

(22) Filed: Oct. 15, 2004

Related U.S. Application Data
(60) Provisional application No. 60/511,218, filed on Oct. 15, 2003.

(51) Int. Cl. A61M 29/00

(52) U.S. Cl. 606/192; 427/2.1; 428/35.7

(57) ABSTRACT

Methods of joining two structures made from dissimilar polymeric materials involve applying a coating containing a polymeric resin, such as a polyurethane-based polymer, to at least a portion of the first structure and welding the second structure to the coated portion of the first structure.

### CROSS-REFERENCE TO RELATED APPLICATIONS

The present application claims the benefit of U.S. Provisional Patent Application Ser. No. 60/511,218 filed Oct. 15, 2003, the disclosure of which is incorporated herein in its entirety by this reference.

### BACKGROUND

1. Technical Field

The present disclosure relates generally to methods for joining structures made of dissimilar polymeric materials, especially when joining dissimilar polymeric materials when making a surgical apparatus, such as access devices, balloon dissectors or other devices that include an elastomeric structure secured to a rigid structure.

2. Background of Related Art

During laparoscopic procedures, cannulas are utilized to provide an access port for surgical instruments and a conduit for introducing insufflation gases into the body cavity. Typically, a trocar is positioned within the cannula and utilized to guide or advance the cannula into the tissue or abdominal wall. Thereafter, the trocar is removed leaving the cannula in place at which time insufflation gas may be forced into the body cavity to form an anatomical operating space. In certain instances, a dissection instrument having a dissection balloon operatively connected to a distal end thereof is inserted into the body cavity. The dissection balloon is inflated to separate the tissue. It is important that a fluid seal is maintained between the dissection balloon and the exterior of the body.

One known balloon dissector has an access cannula with a threaded stabilization device. The threaded stabilization device prevents the cannula from migrating further into or out through the incision. Additionally, the stabilization device also operates as a skin seal, to prevent leakage of insufflation gases.

Balloon anchors on access cannulas are generally known and such balloon anchors are disposed inside the body and inflated. A foam collar is utilized on the exterior of the access cannula to hold the cannula in place, in cooperation with the balloon anchor. The balloon also prevents leakage of insufflation.

Another prior art device, known as a structural balloon trocar (“SBT”), is used to maintain an operating space within a cavity of the body. Such SBT may be used in hernia repair operations, to maintain the operating space and access a hernia. Like the Balloon anchored access cannulas, the SBT includes an insufflation port, for introducing insufflation gases to aid in maintaining the operating space. The SBT also has a foam collar for securing the device and sealing around the incision.

In each of the devices above, a balloon, which is made from a first polymeric material, is attached to a member, which is typically a rigid structure made from a second polymeric material that is different from the first polymeric material. However, most types of thermoplastic joinery can only be performed with like or compatible materials. This presents problems when welding two dissimilar materials with incompatible melt points, durometers, vicat temperatures, etc. Hence, bonding separate thermoplastic components is difficult to achieve.

There remains room for improvement in the techniques used to join dissimilar thermoplastic materials together, such as to produce surgical or medical apparatus.

### SUMMARY

Methods of joining two structures made from dissimilar polymeric materials involve applying a coating containing a polymeric resin, such as a polyurethane-based polymer, to at least a portion of the first structure and welding the second structure to the coated portion of the first structure. The coating can be applied to the first structure by applying a composition containing a polymeric resin (or precursor(s) thereof) and a solvent to at least a portion of the first structure, curing the resin, if necessary, and removing at least a portion of the solvent to leave a coating of the resin on at least a portion of the first structure. In some embodiments, the first structure is made from a biocompatible high strength thermoplastic material, the second structure is made from a biocompatible elastomeric resin, and the coating is made from an elastomeric resin. In particularly useful embodiments, the first structure is made from a polycarbonate, the second structure is made from a polyurethane, and the polymeric resin is a polyurethane-based polymer, such as a polyurethane or an aliphatic polycarbonate-based thermoplastic polyurethane.

According to one embodiment of the present disclosure, there is provided a surgical instrument including a housing having an orifice; a cannula having a proximal end connected to the housing and a distal end, the cannula having a lumen which is in communication with the orifice; and a balloon welded to the cannula. The cannula is coated with a polymeric composition that facilitates welding of the balloon to the cannula. In some embodiments, the surgical instrument further includes one or more attachment members for securing the balloon to the cannula. The one or more attachment members are welded to the coated portion of the cannula, and the balloon is welded to the one or more attachment members. In one embodiment employing a single attachment member, the attachment member is a sleeve disposed on the outer surface of the cannula. In another embodiment employing two attachment members, the attachment members are a first collar and a second collar. The first and second collars each can have a tube portion that is welded to the coated cannula and a flange to which the balloon welded.

The material used to coat the cannula facilitates welding of the balloon to the cannula. The coating material includes a material that is easily weldable to the material from which the balloon is made, such as, for example, an elastomeric resin. Good adhesion of the coating to the cannula is achieved by use of a coating composition containing a solvent in addition to the elastomeric resin composition.

In a particularly useful embodiment, the cannula is made from a polycarbonate material and the surface of the balloon that is secured to the cannula is made from a polyurethane. In this embodiment, the coating is advantageously derived from a urethane slurry or a solution that forms an aliphatic polycarbonate-based thermoplastic polyurethane.

The balloon may include a multilayer material having a first layer of a first polymeric material, a second layer of a second polymeric material and a third layer of a third polymeric material, the second layer being interposed between the first layer and the third layer. Desirably, the first and third polymeric materials comprise polyurethane and the second polymeric material comprises polyester. It is envisioned that the cannula comprises a fourth polymeric material, such as, for example, polycarbonate. The coating composition can include the first material, the third material or a fifth material that exhibits welding compatibility with either the first or third materials.

According to another aspect of the present disclosure, there is provided an access device, for use with surgical instruments. The access device includes a cannula made of a first material and having a distal extremity, a proximal extremity, and defines a lumen therethrough; a coating containing an elastomeric resin on at least a portion of the surface of the cannula; and a balloon made at least in part of a second material that is different from and incompatible with the first material (from which the cannula is made). The balloon is welded to coated portion of the cannula.

It is envisioned that the structural balloon may include a multilayer material having a first layer of a first polymeric material, a second layer of a second polymeric material and a third layer of a third polymeric material, the second layer being interposed between the first layer and the third layer. The cannula is made from a fourth material that is different from and incompatible with the first layer. It is envisioned that at least one of the first and third polymeric materials may be polyurethane. It is further envisioned that the second polymeric material may be a polyester. It is further envisioned that the fourth polymeric material may be a polycarbonate. Desirably, the multilayer material is attached to a portion of the cannula that is coated with an elastomeric resin so that the first layer contacts the coating.

Other objects and features of the present disclosure will become apparent from consideration of the following description taken in conjunction with the accompanying drawings.

### BRIEF DESCRIPTION OF THE DRAWINGS

These and other features, aspects and advantages of the present invention will become better understood with regard to the following description, appended claims and accompanying drawings where:

FIGS. 1A-1C schematically depict the steps of an illustrative embodiment of the methods of joining materials in accordance with the present disclosure;

FIGS. 2A and 2B are schematic side elevational views of a balloon dissector assembly and obturator, respectively, wherein the balloon of the dissector assembly is attached to the cannula using a method in accordance with the present disclosure;

FIG. 3 is a schematic side elevational view of an access device having a balloon attached to the cannula using a method in accordance with the present disclosure;

FIG. 4 is a schematic cross-sectional view of a balloon attachment of the access device in accordance with the embodiment of FIG. 1 taken through 4-4 of FIG. 3;

FIGS. 5A and 5B are perspective views of a collar suitable for use as an attachment member in accordance with one embodiment of the present disclosure;

FIGS. 6A through C show the steps in assembling the balloon assembly and the cannula in accordance with one embodiment of the present disclosure;

FIG. 6D shows the cannula having a balloon assembly mounted thereon as part of an access device in accordance with one embodiment of the present disclosure; and

FIG. 6E is a perspective view of a balloon assembly and cannula in accordance with a further embodiment of the invention.

### DETAILED DESCRIPTION OF PREFERRED EMBODIMENTS

Preferred embodiments of the presently disclosed methods and surgical instruments, including an access device according to the present disclosure, will now be described in detail with reference to the drawings, in which like reference numerals designate identical or corresponding elements in each of the several views. As used herein, the term “distal”, as is conventional, will refer to that portion of the instrument, apparatus, device or component thereof which is furthest from the user while, the term “proximal”, will refer to that portion of the instrument, apparatus, device or component thereof which is closest to the user.

Certain embodiments of the disclosure relate to medical devices having at least two different thermoplastic components. Typically, the medical devices of the present disclosure include a first component of a first biocompatible polymeric material and a second component of a second biocompatible polymeric material that is different from the first polymeric material. In particularly useful embodiments, the first polymeric material is a biocompatible high strength thermoplastic material and the second polymeric material is a biocompatible elastomeric material.

The first polymeric material from which the first component is made is any material that is suitable for construction of the particular medical device being made. Preferably the first polymeric material is a biocompatible high strength thermoplastic material. “High strength” as used herein refers to a thermoplastic material with a Shore A hardness of at least about 90. Suitable high strength thermoplastics include, but are not limited to homopolymers and copolymers of polycarbonate, polyethylene, PEBAX (polyether block amides), polyvinyl chloride (PVC), polyolefin, polystyrene, nylon, polyimide, or other conventional biocompatible high strength thermoplastic materials. Preferably, the first component is a polycarbonate. Polycarbonates include homopolymers and copolymers such as block copolymers. Polycarbonates are well-known as extremely hard and brittle materials. Due to the inherent properties of polyurethanes and polycarbonates, these materials traditionally are not easily joined together via methods such as welding or co-extrusion.

The second component is preferably made from a biocompatible elastomeric resin. There is no particular limitation on the biocompatible elastomeric resin material used to form the second component. Elastomeric resins include, for example, homopolymers, copolymers, polyesters, nylon, and urethanes with a Shore A hardness of less than about 75. Suitable materials include, but are not limited to polyurethane, silicone, latex, epoxy, rubber, soft polyvinyl chloride (PVC), polyolefins such as polyethylene terephthalate (PET), polyethylene (PE), polypropylene (PP), PTFE, polyamide, polystyrene, polyester, nylon, or other suitable biomedically-acceptable elastomers. Preferably, the elastomeric resin is polyurethane. Materials such as polyurethanes have been historically used in medical applications such as balloon catheters because of its pliability, modulus, and elongation characteristics.

In order to facilitate securing the first component to the second component, a polymeric coating is applied to at least a portion of the first component. The polymeric coating can be any polymeric composition that facilitates welding of the first component to the second component. The coating composition can include as a component thereof the same polymer as the second polymeric material (i.e., the material from which the second component is made). Thus, in certain embodiments, the coating includes a biocompatible elastomeric resin material of the type listed above as suitable for use as the second polymeric material. Alternatively, coating can include a block copolymer that includes an elastomeric block. Suitable materials for use in preparing the coating composition in clued the Hi-Touch™ line of thermoplastic elastomers available from Apex Medical Techniologies, Inc., San Diego, Calif., USA, with HT-7 being particularly preferred. Another suitable material for us in formulating the coating composition is a material commercially available under the trade name CARBOTHANE® (available from Thermedics, trademark of Noveon). This material is aliphatic polycarbonate-based thermoplastic polyurethane (TPU).

The coating can be adhesively or chemically bonded to the first component. In certain embodiments, the coating is solvent bonded to the first component.

The coating is provided on the first component by contacting the first component with a coating composition. The coating composition can include a solvent and the polymer to be coated on the first component, or precursors of the coating polymer which can be polymerized or cured to provide the coating polymer. For example, rather than contain a polyurethane polymer, the coating composition can include a polyhydroxy compound and a diisocyanate and after being applied to the first component can be cured in situ to provide a polyurethane coating on the first component. Curing can be achieved using heat, UV, light or any other method. Suitable additional ingredients, such as, for example, initiators, chain extender, plasticizers, etc., may be added to the coating composition as those skilled in the art will appreciate.

The organic solvent used in the slurry of the present disclosure is preferably selected in accordance with the ability of the solvent to prepare a slurry of the polymeric coating material and the ability of the solvent to dissolve at least a part of the surface of the material from which the first component is made. Exemplary solvents include, but are not limited to, organic solvents. For instance, when the elastomeric resin material is a polyurethane, useful organic solvents to form a slurry include ketones, such as acetone, methyl ethyl ketone, diethyl ketone, and methyl isobutyl ketone; esters, such as ethyl formate, ethyl acetate, butyl formate and butyl acetate; halogenated hydrocarbons, such as carbon tetrachloride, chloroform, chlorobenzene, dichloroethane and trichloroethane; aromatic hydrocarbons, such as benzene, toluene and xylene; and cyclic ethers, such as tetrahydrofuran and dioxane. Additionally, mixtures of any organic solvent may also be used. Typically, the organic solvents used are dependent upon both the solubility of the polymeric coating material (or its precursors) and the solubility of the material from which the first component is made. Tetrahydrofuran is a particularly preferred solvent when the first component is made from a polycarbonate and the polymeric coating material is a urethane-containing polymer. Typically, the solvent is present in the coating composition in an amount of from about 5 to about 95 percent by weight of the coating composition.

The first component can be contacted with the coating composition using any technique within the purview of those skilled in the art. Suitable techniques include dipping, spraying and brushing.

In one embodiment, a portion of the first component is dipped into the coating composition and remains therein until the at least a part of the surface of the first component is dissolved. Typically, the amount of time the component is contacted with the coating composition depends upon the solubility of the material from which the first component is made in the particular organic solvent used to formulate the coating composition. An organic solvent in which the first polymeric material has a high solubility decreases the amount of time the component is submerged in the slurry. Contact times can range from 1 second to 10 minutes, preferably from 3 to 10 seconds. Additionally, only a part of the surface of the first polymeric material is dissolved such that the structural integrity of the first component is maintained. The dissolution of at least a part of the surface of the first component creates a bond between the first component and the coating once the volatiles of the organic solvent have been removed. The volatiles can be off-gassed by air-drying at room temperature however, the off-gas may occur at an elevated temperature such as temperatures above about 35° C.

The thickness of the coating is not critical. Typically the thickness of the coating can range between about 0.01 mil to 50 mil, preferably 0.5 to 30 mil. The thickness of the coating can be increased by optionally applying additional layers of the polymeric coating material to the first component.

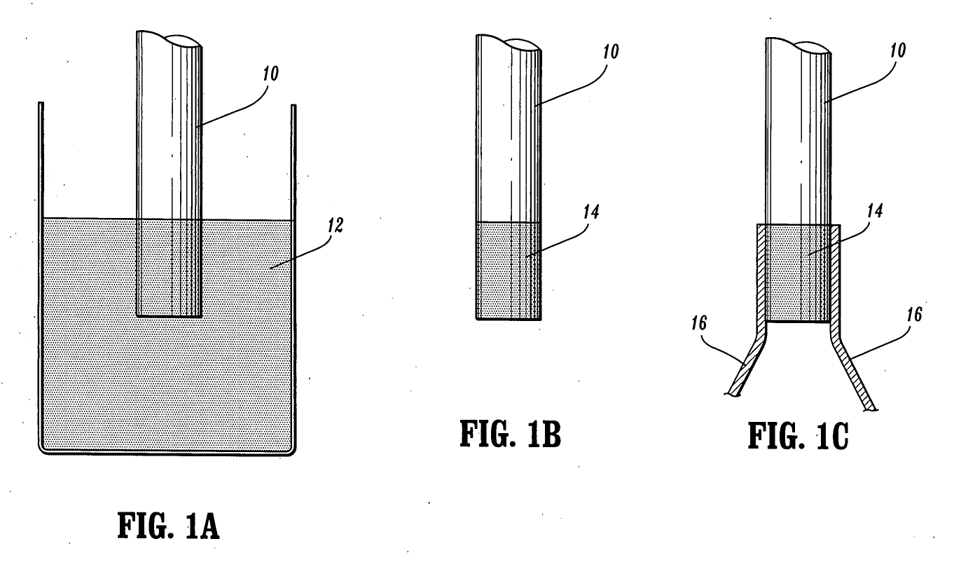

One embodiment of the present disclosure is a process for joining a polyurethane component with a dissimilar material such as a polycarbonate. A process for joining the two materials is schematically shown in FIGS. 1A through 1C. As shown in FIG. 1A, a first component, such as polycarbonate tube 10 is immersed in a solution-grade polyurethane slurry 12. The first component can be, for example, the distal end of a balloon dissector or the distal end of an access cannula. A urethane-containing or urethane-forming material (e.g., HT7, CARBOTHANE® or the like) is combined with an organic solvent (such as tetrahydrofuran) such that a urethane slurry is formed. The urethane slurry 12 coats the polycarbonate tube 10 and the organic solvent partially dissolves the surface of the polycarbonate tube 10. Organic solvents in the urethane slurry 12 cause a chemical reaction that merges the urethane slurry 12 and the polycarbonate tube 10. The bond between the tube 10 and the slurry 12 may be an adhesive bond or chemical bond depending on the material from which the tube 10 is made. The polycarbonate tube 10 is removed from the urethane slurry 12 and dries or cures into a skin of polyurethane. Once the volatiles off-gas, only the polyurethane coating 14 is left behind as shown in FIG. 1B.

Once the polyurethane coating 14 is dried, a second component 16 may be welded to the tube 10 as shown in FIG. 1C. Typically, the second component 16 is made of an elastomeric resin material that is either the same as or different than the polymeric coating material. In an exemplary embodiment of the disclosure, the second component 16 is a polyurethane balloon. Any type of welding (e.g. RF, impulse, etc.) may be used to join the second component with the first component. For instance, laser impulse welding may be used weld the polyurethane balloon to the polycarbonate tube via the polyurethane coating. Hence, the coating of the first component provides a similar surface to which the second component of (e.g., balloon) can be adhered.

Embodiments of the present disclosure provide a method of joining a thermoplastic with a material that is not amenable to welding. Although polycarbonate is used as an example, other materials may be immersed in the urethane slurry. Any material that is susceptible to partial dissolution by the solvent and retains a coating once the solvent volatiles off-gas may be used. A variety of materials may benefit from the process, particularly materials that are non-thermoplastics and not typically weldable.

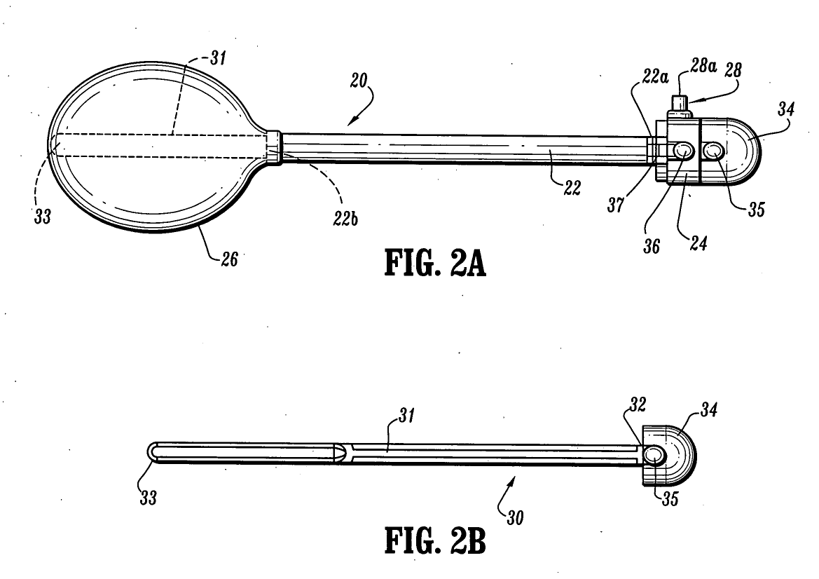

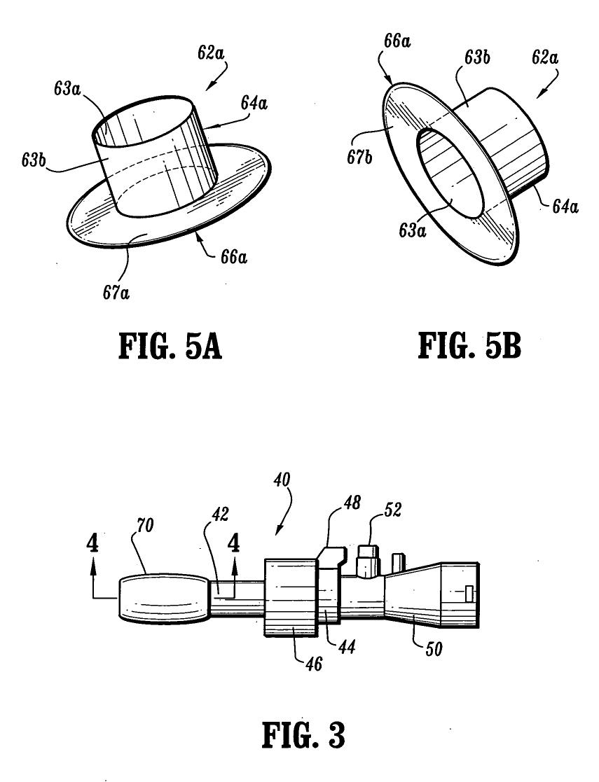

FIG. 2 shows a balloon dissector assembly 20 and FIG. 3 shows an access device 40 that can be made in accordance with the present disclosure. While the following disclosure relates generally to the making and use of access device 40 in combination with a balloon dissector assembly 20 suitable for performing, for example, extraperitoneal hernia repair, it is envisioned and within the scope of the present disclosure that the present methods of joining materials may be used to make other devices including, but not limited to balloon retractors and the like, or any other laparoscopic surgical instrument suitable for performing a variety of other surgical procedures known to one having ordinary skill in the art.

Surgical dissection instruments are used for insertion into the body of a patient to create or enlarge a cavity or anatomic space. As shown in FIGS. 2A and 2B, balloon dissector assembly 20 includes a tubular member 22 having a bore extending therethrough, and an obturator 30 slidably mounted in the bore of the tubular member 22. The obturator 30 includes a proximal extremity 34 and a distal extremity 33 having a blunt tip. The tubular member 22 has a proximal end 22a and a distal end 22b. Tubular member 22 is formed of a rigid plastic material. A housing 24 is operatively connected to the proximal end 22a of tubular member 22. The housing 24 includes at least one internal seal member (not shown) to seal the bore of tubular member in the absence of obturator 30 and while the obturator 30 is disposed within the bore. Reference may be made to U.S. Pat. No. 6,312,442 for a more detailed discussion of the structure and use of a balloon dissector.

Balloon dissector assembly 20 further includes a dissection balloon 26 operatively secured on distal end 22b of tubular member 22. The dissection balloons may have any shape and may be elastic, rigid or inelastic. In particularly useful embodiments, dissection balloon 26 advantageously may be one of two shapes (i.e., round and oval) depending on surgeon preference and patient anatomy. The dissection balloon 26 has an interior and is attached to the tubular member 22 so that the interior of the dissection balloon 26 and the bore of the tubular member are in communication.

As seen in FIG. 2A, balloon dissector assembly 20 further includes a balloon inflation port 28, and a valve assembly 28a connected to the port 28. The valve assembly 28a couples with an inflation device (not shown), e.g., an inflation bulb, for transmission of inflation fluid to dissection balloon 26. The port 28 is in communication with the bore of the tubular member 22 for utilizing inflation bulb in inflating the dissection balloon 26.

As seen in FIG. 2B, the obturator 30 comprises a shaft 31 having a proximal end 32 and a distal end 33. A handle 34 is attached to the proximal end 32 of the shaft 31 and includes buttons 35. Buttons 35 are attached to latches (not shown) for engaging recesses (not shown) in the housing 24 so that the obturator 30 may be secured to the housing 24 to provide the balloon dissector assembly 20. Housing 24 includes buttons 36, which are also attached to latches 37 for assembly of the balloon dissector assembly 20 with the access device 40.

Balloon 26 is attached to the distal end 22b of tubular member 22 in accordance with one embodiment of the present disclosure by first dipping the distal end 22b of tube 22 into a urethane slurry to coat the distal end of tube 22 with a polyurethane coating (as shown schematically in FIGS. 1A and 1B). The balloon 26 can then be welded to the coated portion of tube 22 (as shown schematically in FIG. 1C).

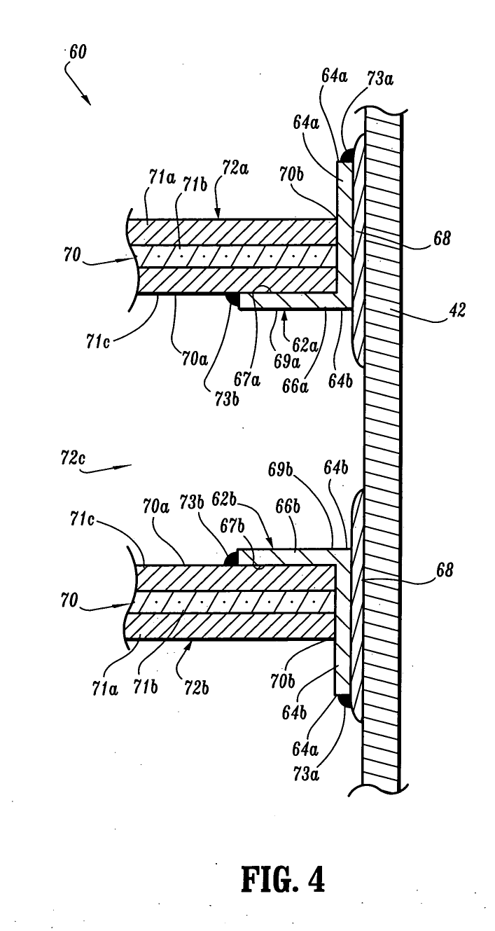

Turning now to FIGS. 3 and 4, access device 40 includes a cannula 42, a locking collar 44 operatively associated with cannula 42, and a foam collar 46 extending distally from locking collar 44. A latch assembly 48 is provided on locking collar 44 to secure the locking collar 44 to the cannula 42. Foam collar 46 is affixed to the locking collar 44 and is compressible against the abdominal wall to provide a secure seal. Reference may be made to International Application Serial No. PCT/US02/17359 for a detailed discussion of the operation and use of latch assembly 48 and foam collar 46. The disclosure of International Application Serial No. PCT/US02/17359 is hereby incorporated by reference herein, in its entirety.

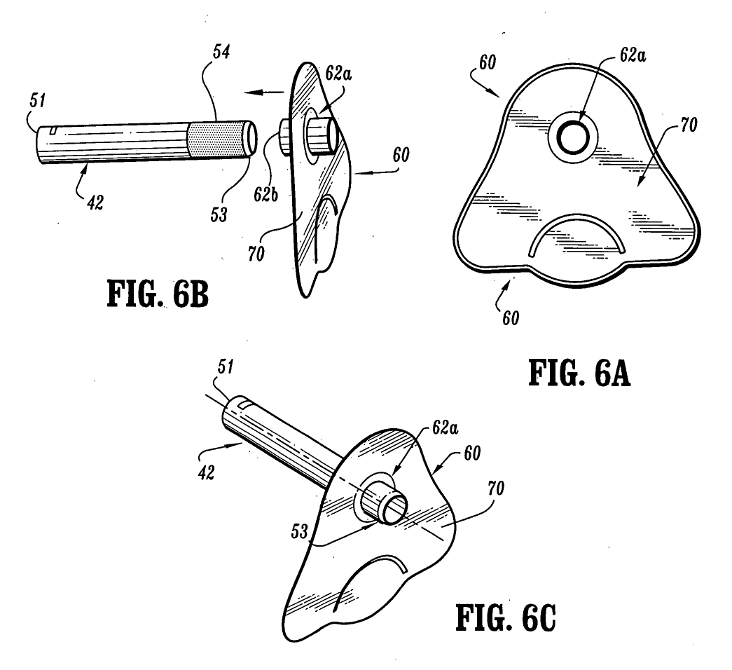

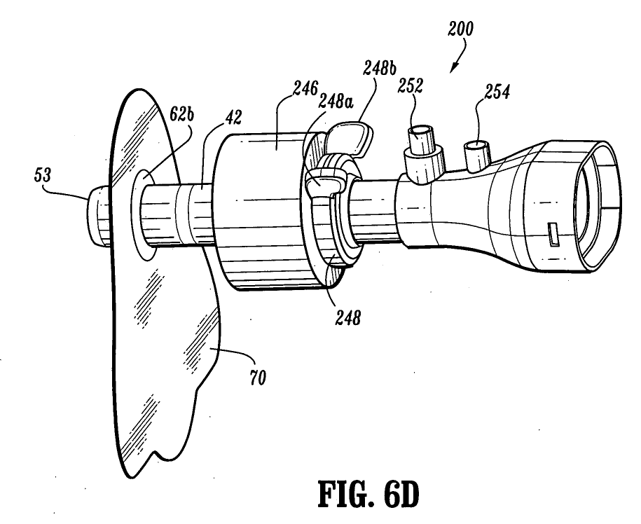

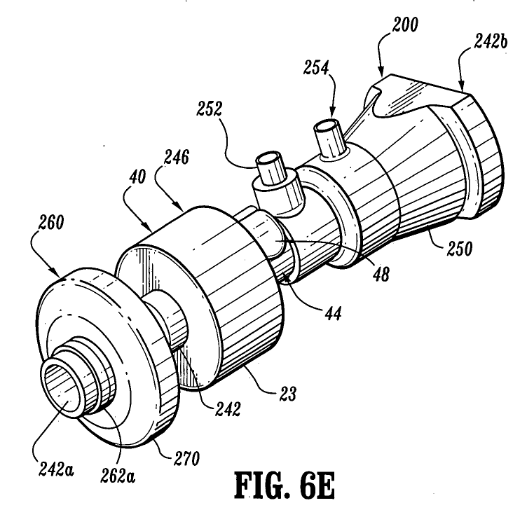

The locking collar 44 may also have a lock incorporating a torsion spring 248 as seen in FIG. 6D, in place of the latch assembly 48. The torsion spring 248 is arranged so that pressing the ends 248a, 248b of the spring together causes the spring to radially expand, allowing the user to slide the foam collar 246 along the cannula 42. When the ends 248a, 248b of the spring 248 are released, the position of the foam collar 246 is secured. A further device for securing the position of the access device is a skin seal having a threaded exterior. Such devices are known and are disclosed, for example, in certain embodiments of U.S. Pat. No. 5,403,336. In further embodiments, a rubber member is slidable along the cannula, and simply frictionally engages the cannula.

Referring to FIGS. 3 and 6B, the cannula 42 has a proximal end 51 and a distal end 53. A housing body 50 operatively connected to a proximal end 51 of cannula 42. Cannula 42 has a tubular wall defining a passageway communicating with an opening in the housing body 50 for receipt of operating instruments therethrough. A balloon assembly 60 is supported on or is otherwise attached to cannula 42 and is in fluid communication with an inflation port 52 provided on housing body 50. A fluid channel is defined within the wall of the cannula 42 and connects inflation port 52 with balloon assembly 60.

Cannula 42 can be made of any rigid material. Suitable material include polymeric materials, such as those identified above for use in making the first component. A particularly useful class of polymeric materials are polycarbonate materials.

As seen in FIGS. 4 and 6A-D, balloon assembly 60 includes a pair of attachment members, namely, first or distal collar 62a and second or proximal collar 62b, each of which is attached to cannula 42. As seen in FIGS. 4, 5A and 5B, each collar 62a, 62b includes a tube portion 64a, 64b, respectively, and a flange 66a, 66b, respectively, extending orthogonally from one another. Additionally, collars 62a, 62b are positioned on cannula 42 such that respective flanges 66a, 66b of collars 62a, 62b are oriented towards one another, or are in juxtaposed relationship, and located in the interior 59 of the balloon 70. Flange 66a defines an inner surface 67a, and an outer surface 67b, and flange 66b has an inner surface 69a, and an outer surface 69b. Tube portion 64a has a cannula side 61a and a balloon side 61b, whereas tube portion 64b has a cannula side 63a and a balloon side 63b.

The collars, although shown in the figures as having a tubular shape with a generally perpendicular depending flange, may have other shapes. For example, the collars may be two separate simple cylindrical sleeves with no depending flanges. As another example, the two collars may be connected as a single sleeve with two spaced apart, depending flanges thereby forming a single attachment member. As another example, a single cylindrical sleeve with no depending flanges may be substituted for the first and second collars as a single attachment member.

Balloon assembly 60 further includes a structural balloon 70 secured to flanges 66a, 66b of collars 62a, 62b. The balloon 70 has an inner surface 70a and an outer surface 70b. In particular, structural balloon 70 is attached to collars 62a, 62b in such a manner that inner surface 70a of structural balloon 70 is secured to the outer surface 67b and 69b of respective flanges 66a, 66b of collars 62a and 62b. However, inner surface 70a may instead be attached to inner surfaces 67a and 69a of the flanges 66a, 66b. Preferably, structural balloon 70 is positioned such that an inner rim 70c of structural balloon 70 is in contact with the balloon sides 61b and 63b of tube portions 64a, 64b of collars 62a, 62b.

As seen in FIG. 4, balloon 70 preferably includes three layers, a first inner layer 71a, a second middle layer 71b, and a third outer layer 71c. In one embodiment, outer layer 71c and inner layer 71a, are fabricated from polyurethane while middle layer 71b is fabricated from polyethylene. It is envisioned that any number of layers may be provided. For example, structural balloon 70 may include two layers, wherein outer layer 71c is removed. Moreover, it is envisioned that layers 71a-71c may be arranged in any order. For example, it is envisioned that middle layer 71b (e.g., the polyethylene layer) is the outer layer of balloon 70. As best seen in FIG. 1, balloon 70 further defines a distal side 72a, a proximal side 72b, and an aperture 72c (See FIG. 6) extending through distal side 72a and proximal side 72b. In certain embodiments, the distal side 72a and proximal side 72b are formed from separate sheets of material welded together at a periphery of the balloon 70. In other embodiments, the balloon 70 is formed from one or more sheets.

The material from which collars 62a, 62b are made is selected to facilitate attachment to balloon 70. For example, the attachment member(s) can be made from a polymeric material the chemical composition of which is compatible from a welding standpoint with polyurethane materials.

In order to secure the collars 62a and 62b to the cannula 42, a portion of the cannula is coated with a polymeric composition 68. In one preferred embodiment, the cannula 42 comprises a polycarbonate material and the balloon 70 comprises polyurethane (and may include layers of the other materials). As those skilled in the art will appreciate, polycarbonate and polyurethane materials are difficult, if not impossible, to weld directly together. In accordance with the present disclosure, therefore, a urethane-containing coating 68 is applied to the cannula. The urethane-containing coating can be applied by contacting cannula 42 with a urethane slurry. A urethane-containing or urethane-forming material (e.g., HT7, CARBOTHANE® or the like) is combined with an organic solvent (such as tetrahydrofuran) such that a urethane slurry is formed. The cannula 42 can be dipped in the slurry of the slurry can be applied to any intermediate portion of the cannul 42 between distal end 53 and proximal end 51, for example by spraying or brushing.

Desirably, as seen in FIG. 4, a first weld 73a is provided between tube portions 64a, 64b of the respective collars 62a, 62b and cannula 42. Preferably, first weld 73a extends along the entire length of each of collars 62a, 62b. Alternatively, weld 73a is a spot or line weld formed along the proximal-most or distal-most edge of collars 62a, 62b around the entire circumference or perimeter of body portion 64 of collars 62a, 62b. Additionally, a second weld 73b is provided between balloon 70 and flanges 66a, 66b of each collar 62a, 62b. Preferably, second weld 73b extends along the entire height of annular flanges 66a, 66b. Alternatively, weld 73b is a spot or line weld formed along the radially outward-most edge of annular flange 66a, 66b around the entire circumference or perimeter of annular flanges 66a, 66b.

First weld 73a maintains the relative axial position of collars 62a, 62b with respect to cannula 42 while second weld 73b maintains the relative position of balloon 70 with respect to each collar 62a, 62b.

In a method of attaching the balloon 70 to the access device 40 a balloon assembly 60 as shown in FIG. 6A is first made. Specifically, one collar 62a is attached to the distal portion 72a of the balloon material and the other collar 62b is attached to the proximal portion 72b of the balloon material by welding the balloon material to the flange 66a, 66b for the respective collar 62a, 62b. Next, the peripheral edges of the distal portion 72a and proximal portion 72b are welded together. The balloon-collar assembly is slid onto the distal end 53 of cannula 42 which has previously been coated with a polymeric coating material (e.g., as described more fully hereinabove) and the tube portions 64a and 64b are welded to the cannula 42 as shown in FIG. 6B at the location of coating 54. It should be understood, of course that the balloon assembly 60 may be secured at the distal end 53 or may be secured at any point along cannula 42 distal of end 53, with the distal end 53 extending distally beyond the balloon 70. FIG. 6C shows the balloon assembly positioned on and secured to the cannula 42.

In a further embodiment of the present disclosure shown in FIG. 6E, the access device 200 comprises a generally toroidal balloon anchor 260 disposed at a distal end 242a of a cannula 242 having a housing 250. The access device 200 includes a foam collar 246 that is slidable along the cannula 242 to cooperate with the balloon anchor 260 in securing the position of the access device 200 in the patient's body. Alternatively, a threaded skin seal or rubber member may be utilized in conjunction with the balloon anchor 260, as discussed above. The housing 250, like housing 50 discussed above, has an inflation port 252 in communication with the balloon anchor 260, and an insufflation port 254 for connection to a source of insufflation gases. A passageway extends through the cannula 242, between distal end 242a and proximal end 242b, for receiving instruments being introduced into the patient's body.

The balloon anchor 260 of access device 200 may be attached to cannula 242 as discussed above in connection with FIG. 4. The balloon anchor 260 comprises a balloon 270 having the shape of a cylindrical sleeve with an aperture extending therethrough, in which the cannula 242 is to be positioned. Each of the proximal end and distal end of balloon 270 are attached to the cannula 242 through one or more collars 262, which are welded to the cannula. For example, a collar 262a for the distal end is shown and a collar 262b for the proximal end is not visible in FIG. 6E. The collars 262 comprise material that is compatible with the material of the cannula 242 and the balloon 270 material for welding, whereas the materials of the cannula and the balloon 260 are not compatible, as described hereinabove.

Although the illustrative embodiments of the present disclosure have been described herein with reference to the accompanying drawings, it is to be understood that the disclosure is not limited to those precise embodiments, and that various other changes and modifications may be affected therein by one skilled in the art without departing from the scope or spirit of the disclosure. All such changes and modifications are intended to be included within the scope of the disclosure.

1. A surgical instrument, comprising:
a polycarbonate tubular member;
a urethane-containing coating on at least a portion of the polycarbonate tubular member; and
a polyurethane balloon welded to the coated portion of the tubular member.

2. A surgical instrument comprising:
a polycarbonate tubular member;
a urethane-containing coating on at least a portion of the polycarbonate tubular member;
at least one attachment member welded to the coated portion of the tubular member; and
a polyurethane balloon welded to the at least one attachment member.

3. A method comprising:
forming a polymeric coating on at least a portion of a first component made from a first biocompatible polymeric material, the coating being made by a) contacting the first component with a coating composition, the coating composition containing a solvent capable of partially dissolving the first biocompatible polymeric material; and b) removing at least a portion of the solvent to form a coated portion on the first component;
welding a second component made from a second biocompatible polymeric material that is different from the first biocompatible polymeric material to the coated portion of the first component.

4. A method as in claim 3 wherein the step of forming a polymeric coating on at least a portion of a first component comprises contacting the first component with a coating composition containing tetrahydrofuran.

5. A method as in claim 3 wherein the step of forming a polymeric coating on at least a portion of a first component comprises contacting the first component with a coating composition containing an aliphatic polycarbonate-based thermoplastic polyurethane.

6. A method as in claim 3 wherein the step of forming a polymeric coating on at least a portion of a first component comprises contacting the first component with a coating composition containing a polyurethane.

7. A method as in claim 3 wherein the step of forming a polymeric coating on at least a portion of a first component comprises contacting a polycarbonate tube with a coating composition.

8. A method as in claim 3 wherein the step of welding comprises welding a polyurethane balloon to the coated portion of the first component.

9. A method as in claim 3 wherein the step of welding comprises welding an attachment member to the coated portion of the first component.

10. A method as in claim 3 wherein the step of contacting comprises dipping the first component into the coating composition.

11. A method comprising:
dipping at least a portion of a polycarbonate tube into a coating composition, the coating composition containing a solvent capable of partially dissolving the polycarbonate tube; and b) removing at least a portion of the solvent to form a coated portion on the tube;
welding a polyurethane balloon to the coated portion of the tube.

12. A method comprising:
applying a polyurethane-containing coating to at least a portion of a polycarbonate tube by contacting the tube with a coating composition, the coating composition containing a solvent capable of partially dissolving the tube; and b) removing at least a portion of the solvent to form a coated portion on the tube;
welding at least one attachment member to the coated portion of the tube.

13. A method as in claim 12 further comprising welding a balloon made at least in part from polyurethane to the at least one attachment member.

14. A method as in claim 12 wherein the at least one attachment member comprises a tubular portion and at least one depending flange.

15. A method as in claim 12 wherein the at least one attachment member comprises first and second collars, each including a tubular portion and a depending flange.

16. A method as in claim 12 wherein the at least one attachment member is made of an aliphatic polycarbonate-based thermoplastic polyurethane.

17. A method as in claim 13 wherein the balloon comprises:
a multilayer material having a first layer of a first polymeric material, a second layer of a second polymeric material and a third layer of a third polymeric material, the second layer being interposed between the first layer and the third layer, at least one of the first or third layers being made of a polyurethane.
